(19) United States
(12) Patent Application Publication
(10) Pub. No.: US 2012/0296240 A1
(43) Pub. Date: Nov. 22, 2012

(54) ULTRASOUND EYE BAG TREATMENT

(75) Inventors: Haim AZHARI, Doar Na Misgav (IL); Liat TSOREF, Tel Aviv (IL); Gregory KRASNOV, Haifa (IL); Solli BRAWER, Kiryat Ono (IL)

(73) Assignee: SLENDER MEDICAL LTD., Herziliya (IL)

(21) Appl. No.: 13/112,393

(22) Filed: May 20, 2011

(51) Int. Cl. A61N 7/00; A61B 17/00

(52) U.S. Cl. 601/2; 606/1

(57) ABSTRACT

A method is provided comprising placing an energy source coupled to a first support structure, at a first location on skin of an eyelid and placing a second structure at a second location on the skin of the eyelid, the second location being farther from the eye than is the first location. A portion of skin of the eyelid and underlying tissue is held between the first and second structures and treatment energy is transmitted from the energy source, from the first location away from the eye, through the tissue, toward the second location on the skin of the eyelid, while withholding transmission of treatment energy from the second location on the skin of the eyelid, toward the eye, through the portion of tissue, toward the first location on the skin of the eyelid. Other embodiments are also described.

### FIELD OF THE APPLICATION

The present invention relates generally to tissue treatment and particularly to methods and apparatus for non-invasive tissue treatment by application of energy thereto.

### BACKGROUND OF THE APPLICATION

Systems for applying energy to biological tissue are well known. Such energy application may be intended to improve the appearance of tissue. Energy may be applied in different forms, such as radiofrequency, laser, microwave, visible and non-visible wavelengths, or ultrasound.

Clinical appearance of a bulge or fullness in the upper or lower eyelids (e.g., eye bags) is typically due to accumulation of excess fat tissue that form fat pads, or adipose pockets, in the eyelids.

Visible effects of eyelid fat pads are disturbing to many individuals and therefore methods for minimizing or removing the fat pads are of interest. Current approaches for treatment of eyelid fat pads include application of natural or synthetic chemical compounds, laser treatments and surgical procedures for modification of the eyelid, e.g., blepharoplasty.

### SUMMARY OF APPLICATIONS

In some applications of the present invention, apparatus and methods for non-invasive cosmetic and/or medical treatment of a tissue are provided. Treatment of the tissue typically includes the application of energy to the tissue for lipolysis of adipose deposits therein. The treatment energy may be applied in different forms, such as radiofrequency, laser, microwave, visible and non-visible wavelengths and/or ultrasound. Applications of the present invention may be applied to treatments such as face-localized removal and/or reduction of adipose tissue. In particular, some applications of the present invention may be applied to treat the area of the eye of a subject, such as upper and/or lower eyelids of the subject, e.g., to minimize eye bags and/or to treat xanthelasma. Additionally or alternatively, some applications of the present invention are applied to treat small lesions, and xanthoma in various areas of the subject's body.

For applications in which the eyelid of the subject is treated, a first support structure is placed at a first location on skin of an eyelid of a subject. The first support structure is coupled to at least one energy source, e.g., an ultrasound transducer, configured to transmit energy to treat the eyelid. A second structure is placed at a second location on the skin of the eyelid, the first location being closer to the eye than the second location. In this context, the first location is referred to herein as the “proximal location,” and the second location is referred to herein as the “distal location.” Tissue (e.g., skin and underlying tissue) of the eyelid is held at least partially between the first and second structures to allow the energy source to treat the tissue that is held therebetween. As provided by some applications of the present invention, the transmission of treatment energy from the energy source is from the proximal location on the skin of the eyelid, away from the eye, through a portion of skin and the underlying tissue, and toward the distal location on the skin of the eyelid. Such transmission of energy is configured to reduce the possibility of treatment energy reaching the eye thereby avoiding causing damage to the eye during the treatment.

For some applications, an acoustic reflector is coupled to the first support structure and is configured to reflect the energy transmitted from the energy source, e.g., the ultrasound transducer, toward a focal zone in the portion of the skin and underlying tissue for treatment of the tissue.

For some applications, eye protection apparatus is provided, for use during application of treatment energy to one or both of the eyelids, or to other tissue. The eye protection apparatus typically comprises a shield configured for placement under an eyelid of a subject being exposed to transmitted ultrasound treatment energy. The shield typically comprises a gas-filled chamber configured to inhibit treatment energy from reaching the eye, by reflecting at least a portion of the transmitted energy.

There is therefore provided, in accordance with some applications of the present invention, a method including:

placing at least one energy source coupled to a first support structure, at a first location on skin of an eyelid of a subject;

placing a second structure at a second location on the skin of the eyelid, the second location being farther from the eye than is the first location;

holding at least a portion of the skin of the eyelid and underlying tissue between the first and second structures; and

causing transmission of treatment energy from the at least one energy source, from the first location on the skin of the eyelid, away from an eye of the subject, through the portion of skin and the underlying tissue, toward the second location on the skin of the eyelid, while withholding causing transmission of treatment energy from the second location on the skin of the eyelid, toward the eye of the subject, through the portion of skin and the underlying tissue, toward the first location on the skin of the eyelid.

For some applications, the eyelid includes an upper eyelid of the subject, and placing the first and second structures includes placing the first and second structures on skin of the upper eyelid.

For some applications, the eyelid includes a lower eyelid of the subject, and placing the first and second structures includes placing the first and second structures on skin of the lower eyelid.

For some applications, placing the first and second structures includes placing the first and second structures on skin of an eye bag.

For some applications, the at least one energy source includes an energy source selected from the group consisting of: a radiofrequency energy source, a laser, an electromagnetic energy source, and a visible wavelength energy source, and causing transmission of the treatment energy from the at least one energy source includes causing transmission of treatment energy from the selected energy source.

For some applications, the energy source includes a probe having a diameter of 2-6 mm, and placing the at least one energy source includes placing the probe at the first location on the skin.

For some applications, a sensor is coupled to the second structure, and the method further includes assessing a parameter of the tissue with the sensor.

For some applications, the sensor includes a temperature sensor, and assessing a parameter of the tissue includes assessing a temperature of the tissue.

For some applications, assessing the parameter of the tissue includes monitoring a parameter selected from the group consisting of: pressure, radiation absorption, and speed of sound.

For some applications, the at least one energy source includes at least one acoustic element, and causing transmission of energy from the at least one energy source includes causing transmission of energy from the at least one acoustic element.

For some applications, causing the transmission of the treatment energy includes causing transmission of the treatment energy as high intensity focused ultrasound (HIFU) energy.

For some applications, a second acoustic element is coupled to the second structure, and the method further includes causing transmission of monitoring energy from the second acoustic element, and monitoring a parameter of the tissue based on the monitoring energy.

For some applications, monitoring the parameter of the tissue includes monitoring a parameter selected from the group consisting of: pressure, temperature, radiation absorption, and speed of sound.

For some applications, an ultrasound reflector is coupled to the second structure, and the method further includes reflecting the treatment energy by the reflector onto a focal zone in the portion of the skin and underlying tissue.

There is further provided, in accordance with some applications of the present invention, apparatus including:

first and second structures configured for placement on skin of an eyelid of a subject and for holding a portion of the skin of the eyelid and underlying tissue of the subject therebetween;

at least one ultrasound transducer coupled to the first structure and configured to transmit ultrasound energy; and

an acoustic reflector coupled to the first structure and configured to reflect the energy transmitted from the ultrasound transducer toward a focal zone in the portion of the skin and underlying tissue.

For some applications, the skin of the eyelid includes skin of an eye bag, and the first and second structures are configured for placement on skin of the eye bag.

For some applications, the acoustic reflector includes a flat acoustic reflector.

For some applications, the acoustic reflector is disposed at a 20-70 degree angle with respect to an axis of transmission of the ultrasound energy by the ultrasound transducer.

For some applications, the acoustic reflector is disposed at a 40-50 degree angle with respect to the axis.

For some applications, the ultrasound transducer is configured to apply the ultrasound energy as high intensity focused ultrasound (HIFU) energy.

For some applications, the apparatus includes a waveguide configured to direct the transmitted ultrasound energy to the reflector.

For some applications, the apparatus includes an acoustic lens configured to focus the ultrasound energy.

For some applications, the reflector is coupled to a portion of the first structure that is configured to be in a path of ultrasound transmission from the ultrasound transducer to the eyelid, during transmission of the ultrasound energy.

For some applications, the ultrasound transducer has a diameter of 5-10 mm.

There is still further provided, in accordance with some applications of the present invention, eye protection apparatus, including:

a shield configured for placement under an eye lid of a subject being exposed to transmitted ultrasound treatment energy, the shield including an outer wall surrounding a gas-filled chamber, the shield configured to inhibit treatment energy from reaching the eye, by reflecting at least a portion of the transmitted energy.

For some applications, the shield is flexible at least in part, and configured to flex to match the shape of the eye.

For some applications, a diameter of the shield is between 1 and 3 cm.

There is additionally provided, in accordance with some applications of the present invention, eye protection apparatus, including:

a shield configured for placement under an eye lid of a subject being exposed to transmitted ultrasound treatment energy, the shield including an outer wall surrounding a foam filled chamber, the shield configured to inhibit treatment energy from reaching the eye, by reflecting at least a portion of the transmitted energy.

The present invention will be more fully understood from the following detailed description of applications thereof, taken together with the drawings, in which:

### BRIEF DESCRIPTION OF THE DRAWINGS

FIG. 1 is a schematic illustration of apparatus for tissue treatment by lipolysis, in accordance with some applications of the present invention;

FIG. 2 is a schematic illustration of another configuration of the apparatus of FIG. 1, in accordance with some applications of the present invention;

FIG. 3 is a schematic illustration of the apparatus of FIG. 1, in accordance with some applications of the present invention;

FIG. 4 is a schematic illustration of structures for holding tissue of the subject therebetween, for applying treatment energy to the tissue, in accordance with some applications of the present invention;

FIG. 5 is a schematic illustrations of the apparatus for tissue treatment, in accordance with some applications of the present invention; and

FIG. 6 is a schematic illustration of eye protection apparatus, in accordance with some applications of the present invention.

### DETAILED DESCRIPTION OF APPLICATIONS

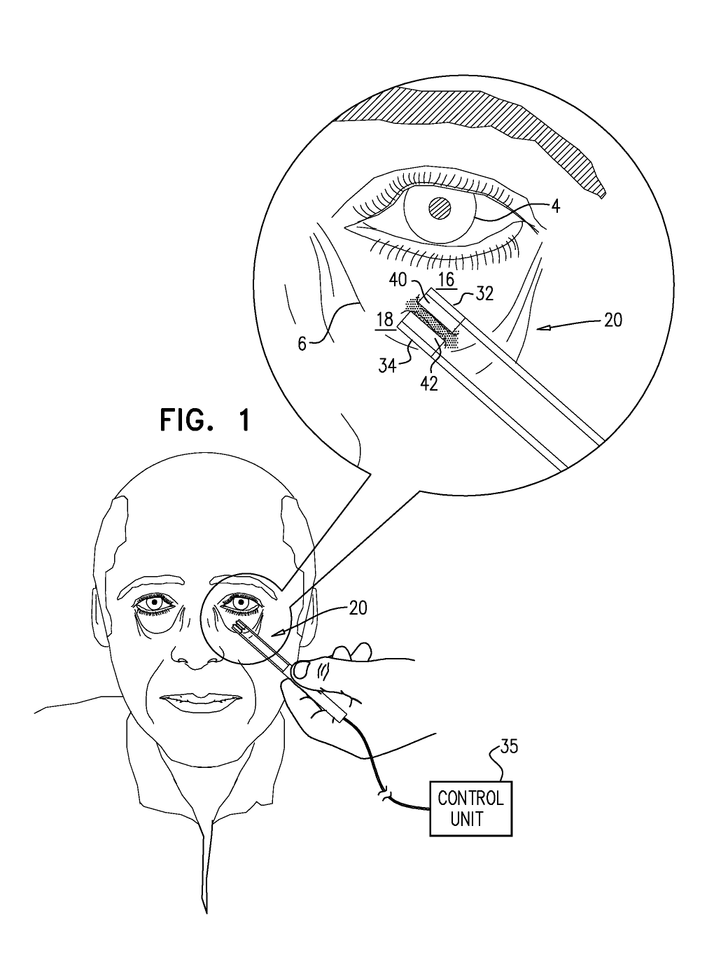

Reference is made to FIG. 1, which is a schematic illustration of apparatus 20 for tissue treatment by lipolysis of fat pads, in accordance with some applications of the present invention. For some applications, apparatus 20 comprises first 32 and second 34 support structures that are configured for placement on skin of a subject, for treatment of the skin and underlying tissue. For some applications, apparatus 20 is configured to treat skin and underlying tissue of delicate and typically small areas in a body of a subject. For example, apparatus 20 may be configured to treat face-localized fat pads. In particular, some applications of the present invention may be applied to treat upper and/or lower eyelids of a subject, e.g., to minimize eye bags and/or to treat xanthelasma.

For applications in which apparatus 20 is used for treatment of an eyelid of the subject, first support structure 32 is placed at a first location on skin of an eyelid of a subject. Second support structure 34 is placed on skin of the eyelid of the subject at a second location, the second location being farther from eye 4 than the first location is from eye 4.

As shown in FIG. 1, apparatus 20 may be used to treat skin of an eye bag 6 of a lower eyelid of the subject. First support structure 32 is placed at a first location 16 on skin of eye bag 6 of the subject. First location 16 is typically closer to eye 4 than is second structure 34, when second structure 34 is placed at a second location 18 on the skin of eye bag 6. A portion of tissue (e.g., skin and underlying tissue) of eye bag 6 is held between the first and second structures. For some applications, the first and second structures 32 and 34 are coupled at one end thereof, and are shaped to define a pinching element, e.g., tweezers or forceps.

First structure 32 is coupled to a treatment element 40. For some applications, treatment element 40 comprises an energy source configured to apply treatment energy to treat the tissue of eye bag 6 that is held between first structure 32 and second structure 34. Typically, the energy is applied from element 40 such that it is focused at a central zone in tissue of eye bag 6. Based or the type of energy source, different forms of energy (such as radiofrequency, laser, ultrasound, electromagnetic and/or optical energy) may be applied to the tissue. Alternatively, or additionally, treatment element 40 comprises a cryogenic treatment element. Typically, treatment element 40 comprises a small probe shaped to define a diameter of between about 2 and 6 mm in contact with the skin. Treatment of tissue of eye bag 6 using the energy source may include, as appropriate, causing heating, tissue damage, thermal ablation, mechanical irritation, cell structure alteration, augmented diffusion, and/or a cavitation effect.

As provided by some applications of the present invention, the transmission of treatment energy from the energy source is from proximal location 16 on the skin of eye bag 6, away from the eye, through a portion of skin and the underlying tissue, and toward distal location 18 on the skin of eye bag 6. Such transmission of energy is configured to reduce the possibility of treatment energy reaching eye 4, thereby avoiding causing damage to the eye during the treatment. It is noted that in some applications, treatment energy is generally not transmitted from the second location on the skin of the eyelid, toward the eye of the subject, through the portion of skin and the underlying tissue, toward the first location on the skin of the eyelid.

It is noted that more than one energy source may be coupled structure 32. Typically, apparatus 20 comprises at least one treatment element 40 coupled to support structure 32 and no treatment element coupled to second structure 34. For some applications, an element 42 is coupled to structure 34 and typically element 42 comprises an element that is not activated to transmit treatment energy.

For some applications, one or both of elements 40 and 42 and/or structures 32 and 34 comprise monitoring functionality, to monitor the effect of the operation of treatment element 40. For example, the monitoring functionality may comprise a temperature sensor. Alternatively or additionally, the monitoring functionality comprises an ultrasound transducer configured to facilitate detection of a change in a parameter (e.g., speed of sound) that is indicative of a change in temperature of the tissue being treated.

For some applications, apparatus 20 comprises monitoring functionality, and element 40 switches from a treatment mode to a monitoring mode. For such applications, elements 40 and 42 are configured to assess a state of tissue of the subject in response to the treatment applied thereto. Typically, elements 40 and 42 are configured to monitor a parameter of the treated tissue, e.g., a physical property such as pressure, temperature, radiation absorption (acoustical, optical or electromagnetic) and/or speed of sound. Based on monitoring of the parameter of the tissue, damage assessment is performed. If appropriate, another iteration of treatment energy is applied by element 40 until satisfactory results are obtained.

For some applications, elements 40 and 42 comprise acoustic elements. Typically, element 40 which is coupled to first support structure 32 comprises an ultrasound transducer which transmits treatment energy through the skin and underlying tissue of eye bag 6 toward element 42. Element 42, which is coupled to second support structure 34, typically comprises an acoustic receiving element designated to receive and/or reflect energy. For some applications, element 42 comprises a reflective element configured to reflect the energy transmitted from the ultrasound transducer towards a focal zone in tissue designated for treatment. For some applications, acoustic element 42 is configured to receive through-transmitted energy and monitor a parameter of the tissue. For such applications, element 42 comprises a transducer which converts the energy into information capable of being processed by a processor typically located remotely from the acoustic elements, enabling the through-transmitted energy to be analyzed, e.g., to calculate a speed-of-sound-based temperature measurement. (Alternatively, no element 42 is provided on second support structure 34.)

Accordingly, first and second support structures 32 and 34 are typically coupled to a control unit 35 that comprises a processor configured to control elements 40 and 42 and to receive and process information obtained by elements 40 and/or 42 for the purpose of monitoring and/or controlling the treatment procedure. Control unit 35 additionally provides, as appropriate, cooling fluid, e.g., liquid or gas, to structures 32 and 34 and elements 40 and 42 via a conduit that extends through structures 32 and 34 from a source of fluid in the control unit.

FIG. 1 shows apparatus 20 placed on skin of an eye bag of a lower eyelid by way of illustration and not limitation. It is noted that apparatus 20 can be used to treat any other region of tissue surrounding the eye, e.g., an upper eyelid. It is noted that applications of the present invention may be applied to treat fat deposits and lipomas in other areas of the body e.g., face, chin, and neck.

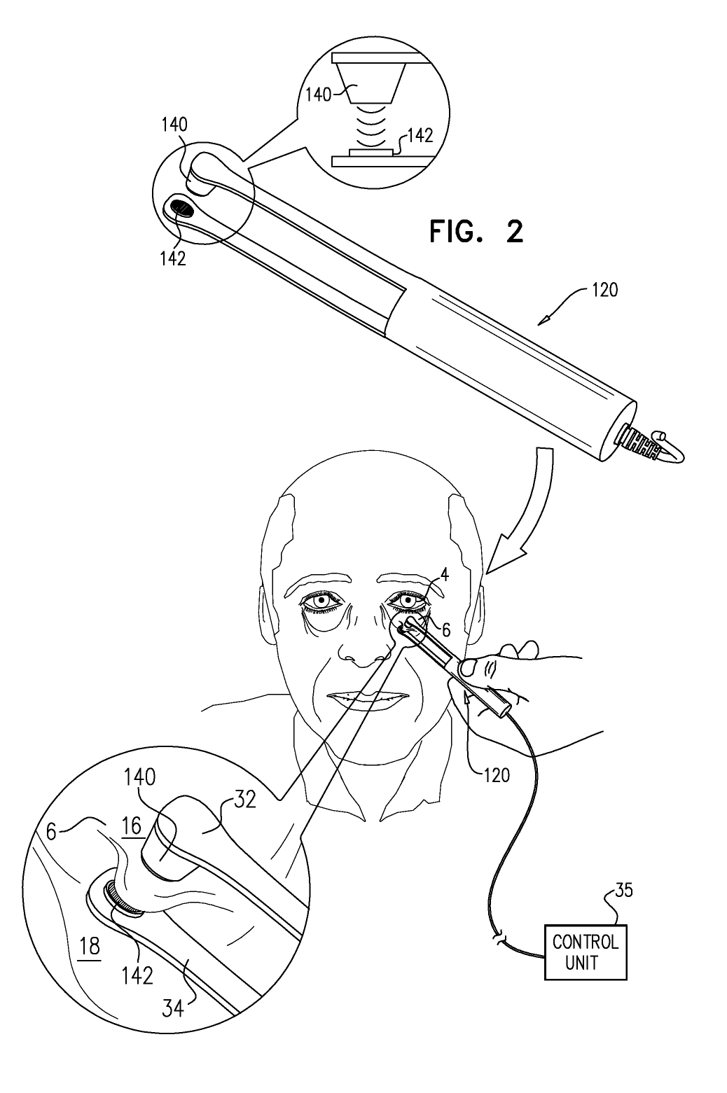

Reference is made to FIG. 2 which is a schematic illustration of apparatus 120, which represents a possible configuration of apparatus 20 described with reference to FIG. 1. Apparatus 120 is similar to apparatus 20 except where stated otherwise. Apparatus 120 comprises first 32 and second 34 support structures that are configured for placement on skin of a subject, for treatment of the skin and underlying tissue. As shown in the exploded view in FIG. 2B, first support structure 32 is placed at a first location 16 on skin of eye bag 6 of a subject. First location 16 is typically closer to eye 4 than is second structure 34, when second structure 34 is placed at a second location 18 on the skin of eye bag 6. A portion of tissue that is designated for treatment (e.g., skin and underlying tissue) eye bag 6 is held between the first and second structures.

For some applications, apparatus 120 is configured to treat tissue of the subject, e.g., by lipolysis of fat pads, by application of ultrasound energy thereto. At least one ultrasound transducer 140 is coupled to first structure 32. Typically, ultrasound transducer 140 comprises a small probe shaped to define a diameter of between about 2-6 mm in contact with the skin. Ultrasound transducer 140 is typically activated to apply treatment energy in the form of high intensity focused ultrasound (HIFU) energy to the tissue of eye bag 6 that is held between first and second structures 32 and 34. Application of high intensity focused ultrasound energy treatment to the tissue may include, as appropriate, causing heating, tissue damage, thermal ablation, mechanical irritation, cell structure alteration, augmented diffusion, and/or a cavitation effect.

Typically, apparatus 120 and structure 32 comprise circuitry for configuring the applied energy as high intensity focused ultrasound (HIFU), using techniques known in the art. For other applications, transducer 140 is configured to apply low intensity focused or non-focused ultrasound energy.

Apparatus 120, e.g., ultrasound transducer 140, is configured to cause transmission of treatment energy from transducer 140 from proximal location 16 on the skin of the eye bag (or eyelid) away from eye 4 of the subject, through the portion of skin and the underlying tissue of eye bag 6, toward the distal location 18 on the skin of the eye bag (or eyelid). Such transmission of energy from a location close to eye 4 towards a location that is remote from the eye is configured to reduce the risk of treatment energy reaching the eye, thereby reducing the possibility of causing damage to the eye during the treatment. It is noted that treatment energy is generally not transmitted from the second location on the skin of the eyelid, toward the eye of the subject, through the portion of skin and the underlying tissue, toward the first location on the skin of the eyelid.

For some applications, a sensing element 142 (e.g. an acoustic sensing element or an electromagnetic sensing element) is coupled to second structure 34. For some applications, sensing element 142 comprises a flat acoustic detecting element that is designated for monitoring purposes and may apply monitoring energy, but is not configured to apply treatment energy to tissue of eye bag 6. Typically, element 142 is configured to assess a state of tissue of a subject in response to the treatment applied thereto. Element 142 is configured to monitor a parameter of the treated tissue, e.g., a physical property such as pressure, temperature, radiation absorption (acoustical, optical or electromagnetic) and/or speed of sound. Based on monitoring of the parameter of the tissue, damage assessment is performed. If appropriate, another iteration of ultrasound treatment energy is applied by transducer 140, until satisfactory results are obtained. For example, acoustic sensing element 142 may comprise an acoustic receiving element designated to receive and/or reflect energy. Element 142 converts the received energy into information capable of being processed by the processor typically located remotely from acoustic element 142 in control unit 35, enabling reflected, scattered, or through-transmitted energy to be analyzed.

Monitoring of the treatment procedure allows an operator of apparatus 120 to monitor the progress of a treatment, and to alter a parameter of the treatment in response thereto. Such a parameter may include, for example, a location of a focus of the HIFU, a positioning of the first and second structure 32 and 34 on the subject's skin, or an intensity of the applied energy.

FIG. 2 shows apparatus 120 placed on skin of an eye bag of a lower eyelid by way of illustration and not limitation. It is noted that apparatus 120 may be used to treat any other region of tissue surrounding the eye, e.g., an upper eyelid. It is also noted, in accordance with some applications of the present invention, apparatus 120 is applied to treat fat deposits and lipomas in other areas of the body e.g., face, chin, and neck.

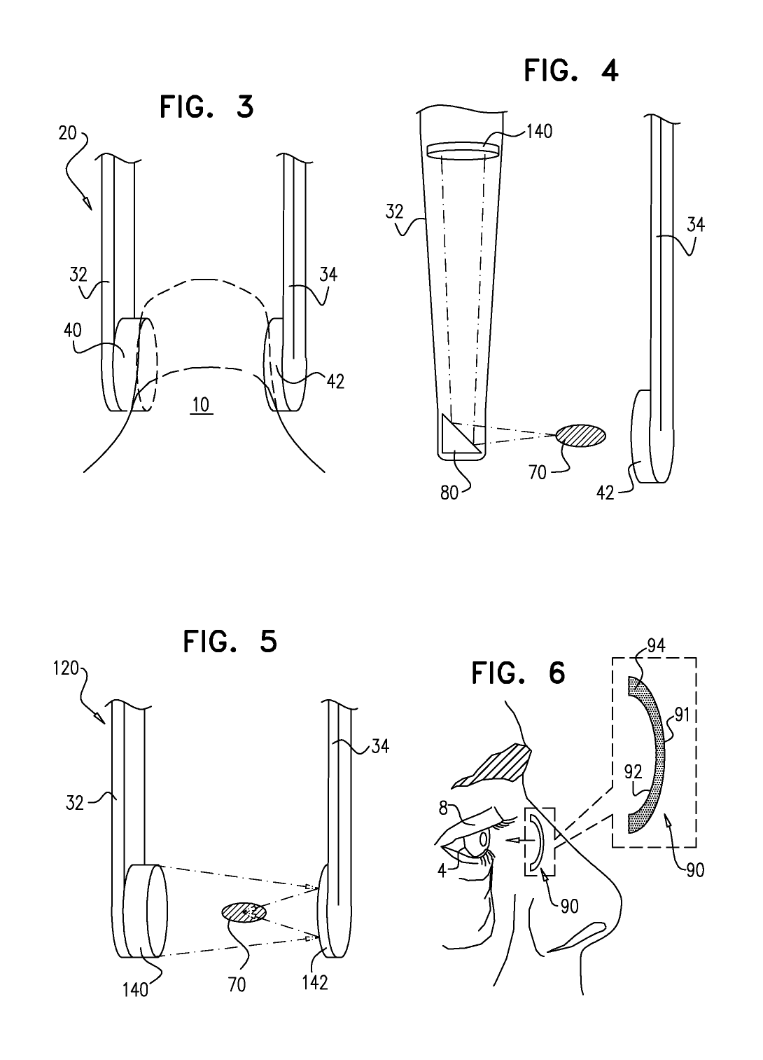

Reference is made to FIG. 3, which is a schematic illustration of support structures 32 and 34 of apparatus 20 in accordance with some applications of the present invention. As shown, a designated area of skin and underlying tissue 10 is held between structures 32 and 34, and treatment energy is applied to the skin and underlying tissue from both or one of elements 40 and 42. Typically, the treatment energy is focused at the central zone in the underlying tissue between elements 40 and 42, so as to reduce damage to the skin. For some applications, one of elements 40 and 42 can be passive throughout the process or at predetermined time points. The treatment energy transmitted (or absorbed in the case of cryogenic treatment) can induce thermal damage or mechanical damage (e.g. through induced cavitation, or shear stress) to tissue region 10 that is shown held between structures 32 and 34.

Reference is made to FIG. 4, which is a schematic illustration of a configuration of support structure 32. For some applications in which element 40 comprises an ultrasound transducer 140, support structure 32 further comprises an acoustic reflector 80 configured to reflect the energy transmitted from ultrasound transducer 140 toward focal zone 70 in tissue designated for treatment. For such applications, ultrasound transducer 140 is positioned within support structure 32 in a location that is farther from the tissue designated for treatment and reflector 80 is positioned in a location that is closer to the treated tissue. Typically, reflector 80 allows the use of an ultrasound transducer 140 that is relatively larger than would be chosen in the absence of a reflector, since the area of the eyelid is small, and it is convenient to locate a larger transducer farther from the actual site of application of the ultrasound energy to the skin. For some applications, ultrasound transducer 140 comprises a piezoelectric element having a diameter of 5-10 mm, and the reflector has an area of 10-80 mm2, and/or a cross-sectional area which is 10-50% of the cross-sectional area of the ultrasound transducer. For some applications, the piezoelectric element is non-circular. For example, the element may be elongated.

For some applications, reflector 80 comprises a flat acoustic reflector. Alternatively, reflector 80 is shaped so as to alter the focal distance of the ultrasound beam originating from transducer 140, or to focus the beam if it is not focused. Reflector 80 is typically positioned before the focus area, such that the ultrasound waves continue their convergence until focal zone 70. Additionally, reflector 80 is positioned at approximately 20-70 degrees, e.g., 40-50 degrees, e.g., 45 degrees with respect to an axis of transmission of the ultrasound energy by the ultrasound transducer (or at another angle, as appropriate), in order to achieve periscope functionality. Typically, reflector 80 is smaller than transducer 140, since the ultrasonic waves partially converge when they reach the reflector, thus allowing placing of reflector 80 within support structure 32 in the relatively small space that is available close to the treated tissue.

For some applications, ultrasound waves originating from transducer 140 are directed to reflector 80 by use of a waveguide, in order to allow curved paths to reach reflector 80 (application not shown). For some applications, a waveguide is used in the absence of reflector 80, to direct ultrasound energy toward the target tissue.

For some applications, an acoustic lens is disposed in acoustic communication between the transducer 140 and reflector 80 or between reflector 80 and focal zone 70, and functions to focus the ultrasound waves. Optionally but not necessarily the acoustic lens may be used in combination with the waveguide.

Reference is made to FIG. 5, which is a schematic illustration of apparatus 120 for tissue treatment, in accordance with some applications of the present invention. As described herein, for some applications, acoustic element 142 is coupled to second structure 34. Acoustic element 142 typically comprises a flat acoustic element that does not transmit treatment energy to tissue of the subject (although, for some applications, the element does transmit energy). Alternatively, element 142 is not flat, e.g., it may be concave.

For some applications, element 142 comprises an acoustic reflector configured to reflect the energy transmitted from ultrasound transducer 140 toward focal zone 70 in tissue of the subject that is designated for treatment, typically such that focal zone 70 is located 3-5 mm away from a surface of transducer 140.

Reference is made to FIG. 6, which is a schematic illustration of eye protection apparatus, in accordance with some applications of the present invention. The eye protection apparatus comprises a shield 90, which is configured for placement under an eyelid 8 of a subject being exposed to transmitted ultrasound treatment energy. Shield 90 is typically placed directly on the subject's eyeball (similar to placement of a contact lens).

Shield 90 typically comprises a posterior surface 92 facing eye 4 of subject, an anterior surface 91 facing away from eye 4. Posterior surface 92 and an anterior surface 91 form an outer wall surrounding gas-filled chamber 94, thereby forming a layer of gas between the posterior and anterior surfaces. Shield 90 is configured to inhibit ultrasound treatment energy from reaching eye 4, by gas-filled chamber 94 reflecting at least a portion of the transmitted ultrasound waves. The gas in the gas-filled chamber 94 is of substantially lower density than the surrounding environment in the eye and in the treated area. The gas has very low acoustic impedance, thus creating a change in acoustic impedance. Due to the change in acoustic impedance, the gas functions as a reflective region, similar to a mirror, and ultrasound waves which reach the gas-filled chamber 94 in shield 90 are reflected away from the eye, thereby protecting eye 4 from potentially damaging ultrasound energy. For some applications, chamber 94 contains a polymeric foam or another foam, which (like the gas) are of lower density than the treated region, and thereby reflect ultrasound energy.

For some applications, shield 90 has an elliptical shape having a major axis of 10-40 mm (e.g., 20-30 mm) and a minor axis of 10-40 mm (e.g., 20-30 mm). For example, the major axis may be 26.5 mm, and the minor axis may be 24.5 mm. As appropriate, the major axis may be 1-30% (e.g., 5-20%) larger than the minor axis. For some applications, shield 90 is circular, and has a diameter that is between 10-40 mm, e.g., 10-30 mm. Shield 90 is shaped to define rounded edges to facilitate placement of shield 90 under eyelid 8 and is typically flexible so as to assume the natural curvature of the eyeball. Typically, shield 90 is composed of a biocompatible material and is sterile and disposable. For some applications, shield 90 comprises a methyl methacrylate resin (e.g., Lucite). For some applications, a suction cup (e.g., a rubber suction cup) is used to facilitate placement and removal of shield 90. Alternatively or additionally, shield 90 is flexible, e.g., to allow easy placement and removal by the physician.

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

1. A method, comprising:
placing at least one energy source coupled to a first support structure, at a first location on skin of an eyelid of a subject;
placing a second structure at a second location on the skin of the eyelid, the second location being farther from the eye than is the first location;
holding at least a portion of the skin of the eyelid and underlying tissue between the first and second structures; and
causing transmission of treatment energy from the at least one energy source, from the first location on the skin of the eyelid, away from an eye of the subject, through the portion of skin and the underlying tissue, toward the second location on the skin of the eyelid, while withholding causing transmission of treatment energy from the second location on the skin of the eyelid, toward the eye of the subject, through the portion of skin and the underlying tissue, toward the first location on the skin of the eyelid.

2. The method according to claim 1, wherein the eyelid includes an upper eyelid of the subject, and wherein placing the first and second structures comprises placing the first and second structures on skin of the upper eyelid.

3. The method according to claim 1, wherein the eyelid includes a lower eyelid of the subject, and wherein placing the first and second structures comprises placing the first and second structures on skin of the lower eyelid.

4. The method according to claim 3, wherein placing the first and second structures comprises placing the first and second structures on skin of an eye bag.

5. The method according to claim 1, wherein the at least one energy source includes an energy source selected from the group consisting of: a radiofrequency energy source, a laser, an electromagnetic energy source, and a visible wavelength energy source, and wherein causing transmission of the treatment energy from the at least one energy source comprises causing transmission of treatment energy from the selected energy source.

6. The method according to claim 1, wherein the energy source includes a probe having a diameter of 2-6 mm, and wherein placing the at least one energy source comprises placing the probe at the first location on the skin.

7. The method according to claim 1, wherein a sensor is coupled to the second structure, and wherein the method further comprises assessing a parameter of the tissue with the sensor.

8. The method according to claim 7, wherein the sensor includes a temperature sensor, and wherein assessing a parameter of the tissue comprises assessing a temperature of the tissue.

9. The method according to claim 7, wherein assessing the parameter of the tissue comprises monitoring a parameter selected from the group consisting of: pressure, radiation absorption, and speed of sound.

10. The method according to claim 1, wherein the at least one energy source includes at least one acoustic element, and wherein causing transmission of energy from the at least one energy source comprises causing transmission of energy from the at least one acoustic element.

11. The method according to claim 10, wherein causing the transmission of the treatment energy comprises causing transmission of the treatment energy as high intensity focused ultrasound (HIFU) energy.

12. The method according to claim 10, wherein a second acoustic element is coupled to the second structure, and wherein the method further comprises causing transmission of monitoring energy from the second acoustic element, and monitoring a parameter of the tissue based on the monitoring energy.

13. The method according to claim 12, wherein monitoring the parameter of the tissue comprises monitoring a parameter selected from the group consisting of: pressure, temperature, radiation absorption, and speed of sound.

14. The method according to claim 12, wherein an ultrasound reflector is coupled to the second structure, and wherein the method further comprises reflecting the treatment energy by the reflector onto a focal zone in the portion of the skin and underlying tissue.

15. Apparatus, comprising:
first and second structures configured for placement on skin of an eyelid of a subject and for holding a portion of the skin of the eyelid and underlying tissue of the subject therebetween;
at least one ultrasound transducer coupled to the first structure and configured to transmit ultrasound energy; and
an acoustic reflector coupled to the first structure and configured to reflect the energy transmitted from the ultrasound transducer toward a focal zone in the portion of the skin and underlying tissue.

16. The apparatus according to claim 15, wherein the skin of the eyelid includes skin of an eye bag, and wherein the first and second structures are configured for placement on skin of the eye bag.

17. The apparatus according to claim 15, wherein the acoustic reflector comprises a flat acoustic reflector.

18. The apparatus according to claim 15, wherein the acoustic reflector is disposed at a 20-70 degree angle with respect to an axis of transmission of the ultrasound energy by the ultrasound transducer.

19. The apparatus according to claim 18, wherein the acoustic reflector is disposed at a 40-50 degree angle with respect to the axis.

20. The apparatus according to claim 15, wherein the ultrasound transducer is configured to apply the ultrasound energy as high intensity focused ultrasound (HIFU) energy.

21. The apparatus according to claim 15, further comprising a waveguide configured to direct the transmitted ultrasound energy to the reflector.

22. The apparatus according to claim 15, further comprising an acoustic lens configured to focus the ultrasound energy.

23. The apparatus according to claim 15, wherein the reflector is coupled to a portion of the first structure that is configured to be in a path of ultrasound transmission from the ultrasound transducer to the eyelid, during transmission of the ultrasound energy.

24. The apparatus according to claim 15, wherein the ultrasound transducer has a diameter of 5-10 mm.

25. Eye protection apparatus, comprising:
a shield configured for placement under an eye lid of a subject being exposed to transmitted ultrasound treatment energy, the shield comprising an outer wall surrounding a gas-filled chamber, the shield configured to inhibit treatment energy from reaching the eye, by reflecting at least a portion of the transmitted energy.

26. The apparatus according to claim 25, wherein the shield is flexible at least in part, and configured to flex to match the shape of the eye.

27. The apparatus according to claim 25, wherein a diameter of the shield is between 1 and 3 cm.

28. Eye protection apparatus, comprising:
a shield configured for placement under an eye lid of a subject being exposed to transmitted ultrasound treatment energy, the shield comprising an outer wall surrounding a foam filled chamber, the shield configured to inhibit treatment energy from reaching the eye, by reflecting at least a portion of the transmitted energy.
